(19) United States
(12) Patent Application Publication
(10) Pub. No.: US 2015/0182483 A1
(43) Pub. Date: Jul. 2, 2015

(54) GLYCATION OF L-LYSINE TO LOWER BLOOD GLUCOSE AND TREAT COMPLICATIONS OF DIABETES

(71) Applicant: Joel Steven Goldberg, Hillsborough, NC (US)

(72) Inventor: Joel Steven Goldberg, Hillsborough, NC (US)

(21) Appl. No.: 14/182,011

(22) Filed: Feb. 17, 2014

Related U.S. Application Data
(60) Provisional application No. 61/923,073, filed on Jan. 2, 2014.

(51) Int. Cl. A61K 31/198

(52) U.S. Cl. CPC A61K 31/198

(57) ABSTRACT

Control of blood glucose is a fundamental goal in the treatment of diabetes mellitus. Glycation of l-lysine can lower blood glucose and advanced glycation end product (AGE) free adducts can be excreted in the urine. Lysine, an inexpensive amino acid, can be administered orally or parentally, and can be incorporated into a stereocomplex matrix as a time released preparation. In patients who suffer from diabetes and concomitant renal insufficiency who cannot clear sufficient quantities of AGE free adducts enantiomeric mixtures of lysine will lower blood glucose and AGEs since d-lysine has been shown to decrease protein glycation and lower hemoglobin Alc. This invention is not intended to replace insulin or oral hypoglycemic agents but is does offer patients a low cost and probably safe complementary therapy for the treatment of diabetes mellitus. In addition to the treatment of diabetes and its complications, this invention could lower AGE burden and slow or ameliorate some of the consequences of aging.

### CROSS-REFERENCES TO RELATED APPLICATIONS

This application claims the benefits of U.S. Provisional Patent Application No. 61/923,073 filed Jan. 2, 2014 each of which is incorporated herein by reference in its entirely.

### FEDERALLY FUNDED RESEARCH

Not applicable

### BACKGROUND OF THE INVENTION

It is well known that decoctions (teas) prepared from the leaves of many plant species will lower blood glucose in diabetic animal models and in humans. (Deguchi & Miyazaki, 2010; Mudra, Ercan-Fang, Zhong, Fume, & Levitt, 2007; Umar, Moh'd, & Tanko, 2013) In Mexico approximately 269 plant species are used to control blood glucose in individuals afflicted with diabetes mellitus. (Hernandez-Galicia et al., 2002) In India leaf extracts of nine plants have been used by tribal communities to treat diabetes. (Arya, Abdullah, Haerian, & Mohd, 2012) Decoctions made from the leaves of Ficus carica (common fig) have been used where the plant is indigenous to lower blood glucose.

A common mechanism to explain the hypoglycemic effects from decoctions of this diverse group of plants has not been elucidated. However, because the teas are often prepared with boiling water it is unlikely that the active ingredient is a protein or any other class of compounds that may be heat labile. It is well known that extractions made from the leaves of many different plants contain significant quantities of amine donor amino acids such as l-lysine. (Lugg & Weller, 1948) L-lysine can be absorbed through the digestive tract, enter the blood, and react with open-chain (chain) glucose forming a Schiff base and as such, decrease the quantity of cyclic glucose by shifting the equilibrium reaction in favor of chain glucose. (FIG. 1) Cyclic blood glucose is the usual form of glucose that is measured in clinical laboratories. The non-enzymatic reaction of glucose with an amine donor is spontaneous proceeding with a rate depending on a multitude of factors foremost of which may be temperature. Glycation of l-lysine in the teas prepared from a wide variety of plant leaves could be the mechanism to explain blood glucose lowering effects from these decoctions.

Various amino acids in particular l-lysine are known to lower blood glucose when administered intravenously or orally, but therapeutic use of l-lysine to lower blood glucose has not been accepted in traditional medical practice. Table 1 shows the prior art.

TABLE 1
Hypoglycemic effects of l-lysine

| Source | Agent | Response |
|---|---|---|
| WO 1999010002 A1 | Rice bran | Diabetes treatment |
| EP 1885293 A2 | L-lysine | Wellness and obesity |
| US20090018196 | L-lysine and other | Lower blood glucose |
| | amino acids | |
| U.S. Pat. No. 7,763,706 | L-lysine and | Diabetes treatment |
| | arginine | |
| WO 1993004690 A1 | L-lysine-histidine | Diabetes treatment |
| WO 2002049636 A1 | L-lysine and other | Diabetes treatment |
| | amino acids | |
| Kalogeropoulou, D. et al. | L-lysine | Lower blood glucose |
| (Kalogeropoulou, | | |
| LaFave, Schweim, | | |
| Gannon, & Nuttall, | | |
| 2009) | | |

In an animal model of chemical diabetes induced by streptozotocin, d-lysine has been shown to decrease the non-enzymatic glycation of hemoglobin, lens proteins and serum proteins and lower hemoglobin A1. (Sensi et al., 1993) Table 2 shows this prior art. (Sensi et al., 1993)

TABLE 2
D-lysine glycation of proteins (Sensi et al., 1993)

| | D-lysine treatment | | |
|---|---|---|---|
| | with streptozotocin | Streptozotocin | |
| | diabetes | diabetes | Control |
| Blood glucose | 25.1 | 22.8 | 5.5 |
| (mmol/l) | | | |
| HbA1 (%) | 3.0 | 4.0 | 2.6 |
| Glycated serum | 1.4 | 2.5 | 1.3 |
| proteins (%) | | | |
| Glycated lens proteins | 4.90 | 5.98 | 3.5 |
| (%) | | | |
| Urinary d-lysine | 15.9 | 0.3 | 0.2 |
| (mmol/l) | | | |

The absorption of oral amino acids including l-lysine is not predictable and is dependent upon amino acid transporters. None of the previously cited sources discuss the subcutaneous or transmucosal administration of l-lysine in a controlled release preparation, as in this invention, as a safe and inexpensive treatment for long term management of diabetes and its complications. Furthermore the prior art does not address the beneficial effects of l-lysine glycation and urinary excretion of deleterious advanced glycation end product (AGE) free adducts.

In patients with normal renal function administration of l-lysine can lower blood glucose and the AGE free adducts formed with l-lysine are cleared in the urine at a rate likely greater than formation of AGE crosslinks. (Bohlender, Franke, Stein, & Wolf, 2005) Some of the more commonly known advanced glycation end products (AGEs) are carboxymethyllysine, pentosidine and glycosepane. Glycosepane is known to deleteriously crosslink collagen and this process may accelerate diabetic complications and aging. (Monnier et al., 2014) Other deleterious effects of AGEs are caused by binding to receptors of advanced glycation end products (RAGE). However, in diabetic patients treated with l-lysine who have compromised renal function elimination of AGEs free adducts may not keep up with crosslink formation. In these patients it is predicted that administration of enantiomeric mixtures of lysine or co administration of d-lysine and l-lysine will decrease blood glucose and likely decrease the formation of AGE crosslinks. (Sensi et al., 1993)

### DRAWINGS

FIG. 1 shows spontaneous and non-enzymatic formation of a Schiff base with an Amadori rearrangement when chain glucose reacts with an amino acid such as d or l lysine. Label 1 is cyclic glucose, label 2 is chain glucose reacting with lysine, label 3 is the Schiff base, and label 4 is the compound formed from the irreversible Amadori rearrangement of the Schiff base.

### DETAILED DESCRIPTION OF THE INVENTION

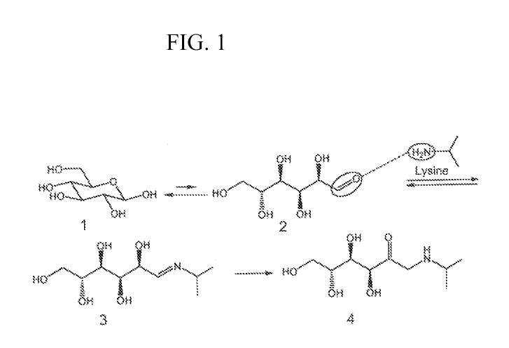

Major mechanisms to lower blood glucose include diet, exercise and administration of insulin and oral hypoglycemic agents. The clinical utility of lowering blood glucose by glycating amino acids has not has not been reported. Even though the concentration ratio of chain glucose to cyclic glucose is very small, glycation in the form of glycated hemoglobin may be viewed as a homeostatic mechanism to lower the quantity of cyclic glucose by shifting the cyclic to chain glucose equilibrium in favor of a decrease in the cyclic form. When lysine glycation occurs, progression to the Amadori product is irreversible so one can view lysine glycation as a constant sink in the cyclic←→chain glucose equilibrium. (FIG. 1)

The formation of the Schiff base is nucleophilic addition at the carbonyl group of chain glucose with elimination of water to form the C═N bond. (FIG. 1) A carbonyl group is not present in cyclic glucose or in any of its isomers. All of the oxygen atoms in cyclic glucose are found as alcohols except for the single oxygen atom bound to two carbons that defines the cyclic structure. (FIG. 1) Therefore formation of AGEs with glucose (produced without intermediates formed by enzymes) begins with chain glucose. However, the final product through the Amadori rearrangement may be cyclic or chain. Lysine glycation is a non-enzymatic method to lower blood glucose forming low molecular weight AGE free adducts that are subsequently excreted in the urine.

L-lysine is the Preferred Amino Acid for Lowering Blood Glucose through Glycation

Nature has shown us that l-lysine is the preferred amino acid to glycate since glycosylated hemoglobin, glucosepane, carboxymethyllysine and pentosidine are formed with glycation of l-lysine.

Charge Considerations that Favor Glycation of L-lysine

In order for non-enzymatic glycation to proceed, the spontaneous nucleophilic elimination reaction requires an uncharged amine as a nucleophile. Though amino acid glycation can occur with many amino acids, at plasma pH of 7.4 most amino acids exist as zwitterions. Only small concentrations of non-protonated amines are present in these amino acids at normal body pH. L-lysine, as an amine donor amino acid, contains two amine groups. Therefore glycation of l-lysine has a greater probability to occur when compared to amino acids with a single amine group.

### Steric Considerations

Of the many amino acids that can form a Schiff base with chain glucose, l-lysine is sterically favored because it contains a second amine donor that is distant to the amine bonded to the alpha carbon. This distance allows non-enzymatic d-lysine glycation with formation of d-enantiomers that can competitively inhibit AGEs comprised of l-lysine as discussed in [0028]. In the two other commonly found donor amino acids namely, arginine and histidine, the donor amine group is hindered compare to that of l-lysine.

### Bioavailability, Lipid Solubility and Isoelectric Point (pI)

Of the various charged species of l-lysine, those with a net charge of zero will have, the greatest transmucosal bioavailability and will cross the lipid membranes from a depot injection into the plasma. When the pH equals pI, at approximately 9.7, 1-lysine is comprised of two predominant microspecies each of which has a net charge of zero. In a reported amino acid (niflumic acid) the non-charged microspecies is 390 times as lipophilic as its zwitterion protonation isomer and such a relationship is expected to exist with l-lysine. (Mazak, Kokosi, & Noszal, 2011)

### Toxicity of L-lysine

The LD 50 dose of l-lysine, an essential amino acid has not been firmly established, but an upper limit of 300-400 mg×kg⁻¹xd⁻¹ has been proposed in humans. (Tome & Bos, 2007) L-lysine has been orally administered in large doses to patients who suffer from herpetic lesions and in subjects with and without concomitant glucose administration. (Flodin, 1997; Griffith, Walsh, Myrmel, Thompson, & Behforooz, 1987) Intravenous l-lysine has been administered to subjects without complications and lowers blood glucose, without significantly increasing blood ammonia. (Kato, Sano, & Mizutani, 1987)

### Stereocomplex Time Release L-lysine

L-lysine can be incorporated into a stereocomplex matrix and time released. Polylactide stereocomplexes are known to safely degrade within the body. The technology for incorporation of small molecules within lactic acid polymers has been described (Stereocomplex polymeric carriers for drug delivery U.S. Pat. No. 6,365,173 B1, Jan. 14, 1999) and this technology may soon become non-proprietary such that these time release preparations can be manufactured without royalties. (Abraham J Domb, 1999)

### Calculating Effects of L-lysine Glycation on Blood Glucose Level

Assuming a blood glucose level of 200 mg/dl in the plasma and a plasma volume of 3 liters and no exchange with the extravascular space:

200 mg/dl×3 liters=6 g glucose

6 g glucose×1 mole/180 g=0.032 moles of glucose in plasma

L-lysine required to lower blood glucose by 50% assuming complete glycation:

0.016 moles l-lysine×146 g/mole=2.2 g l-lysine

### Cost Considerations

At the time of this writing, the cost of 100 g of pharmaceutical grade amino acids from Sigma-Aldrich Corporation (St. Louis, Mo.) is shown in Table 3. L-lysine is one of the lower cost amino acids.

TABLE 3
Costs of amino acids

| Amino acid | Price (USD) |
|---|---|
| L-alanine | 222 |
| L-arginine | 116 |
| L-aspartic acid | 126 |
| L-cysteine | 180 |
| L-glutamic acid | 98 |
| L-glutamate | 85 |
| L glycine | 25 |
| L-glutamine | 85 |
| L-histidine | 447 |
| L-isoleucine | 315 |
| L-leucine | 237 |
| L-lysine | 99 |
| L-methionine | 159 |
| L-phenylalanine | 199 |
| L-proline | 222 |
| L-serine | 610 |
| L-tryptophan | 197 |
| L-valine | 87 |

### Advanced Glycation End Products (AGEs)

AGEs such as glucosepane, known to crosslink collagen, may be responsible for many of the long term complications of diabetes as well as aging. Table 4 shows some AGEs as risk factors in assayed skin biopsy specimens as part of the Diabetes Control and Complications Trial. (Monnier et al., 2014)

TABLE 4
AGEs risk factors

| Retinopathy | Nephropathy | Neuropathy |
|---|---|---|
| furosine | furosine | glucosepane |
| carboxymethyllysine | carboxymethyllysine | fructose-lysine |
| glucosepane | glucosepane | |
| fructose-lysine | fructose-lysine | |

No method to date with clinical utility has been developed to prevent the collagen crosslink produce by glucosepane or other deleterious effects of AGEs. These methods include nucleophilic traps for carbonyl intermediates such as aminoguanidine and putative crosslink breakers such as alagebrium. (Bohlender et al., 2005) In this invention administration of l-lysine can lower blood glucose by glycation of chain glucose and AGE free adducts with relatively low molecular weight are filtered and excreted in the kidney in patients who have normal renal function. (Bohlender et al., 2005) In animal or human studies there is no evidence to suggest that administration of l-lysine will increase AGEs since urinary clearance is likely greater than the formation of AGEs crosslinks.

Treatment of Patients with Diabetes and Renal Insufficiency

In patients who have diabetes with renal insufficiency clearance of AGEs may not be sufficient to prevent the potential increase in AGE free adducts and crosslinks. In these patients administration of l-lysine will decrease blood glucose but may increase the risk of AGEs burden. Therefore in a subset of diabetic patients with renal insufficiency, as defined by elevation of serum creatinine, the preferred adjunctive lysine therapy is an enantiomeric mixture of lysine. Prior art in an animal model establishes that glycation of proteins are decreased in animals administered d-lysine. (Sensi et al., 1993) A possible mechanisms for this observation is that d-lysine competes for l-lysine covalent bonding sites on chain glucose and d-enantiomers of AGEs do not crosslink proteins. It is predicted based upon the general non-reactive characteristics of d-amino acids that crosslinking of d-enantiomeric AGEs (those that contain d-lysine) will be non-physiologic. That is, the crosslinking of d-enantiomeric AGEs with proteins including collagen will not occur or will be weak. Furthermore receptors that are activated by receptor activated end products (RAGE) will not be physiologically activated by d-enantiomers of AGEs. This prediction is in complete agreement with the results of prior art. General applications would be to incorporate constituents of opposite chirality into natural substances to form competitive or non-competitive antagonists.

Properties of D-lysine for the Treatment of Diabetes in Patients with Impaired Renal Function

D-lysine shares many of the same characteristics as l-lysine. Therefore charge considerations [0014], steric considerations [0016], bioavailability, lipid solublility and pI considerations [0018] and stereocomplex time release preparations [0022] are expected identical to those of l-lysine. The toxicity and hypoglycemic effects of d-lysine in humans is not known, but in an animal model d-lysine is excreted in the urine and is not nephrotoxic. (Sensi et al., 1993)

### Benefits to Society

World health care costs for the treatment of diabetes and its complications is approximately 400 billion USD. (Zhang et al., 2010) New agents to control blood glucose and advanced technologies such as insulin pumps with or without real time monitoring of blood glucose are expensive. Data suggest that tight control of blood glucose ameliorates some of the complications of diabetes including ketoacidosis and hypoglycemic coma as well as vasculopathy, retinopathy, nephropathy and neuropathy. Through non-enzymatic glycation, l-lysine, a relatively non-toxic and inexpensive amino acid, can lower blood glucose and its AGE free adducts can be excreted in the urine. L-lysine can be incorporated into polylactide or similar stereocomplexes as a sustained release formulation that can be implanted subcutaneously. This technology may soon be non-proprietary and therefore may be manufactured at low cost. It is not expected that this invention will replace the administration of insulin or oral agents such as metformin but it may provide a safe, effective and inexpensive adjunctive method to modulate blood glucose and improve the lives of patients who suffer diabetes mellitus.

A predicted corollary of this invention is that in select patients with diabetes and renal insufficiency administration of d-lysine or enantiomeric mixtures of lysine can lower formation of AGE crosslinks and inhibit RAGE thereby slowing the rate of diabetic complications and possibly improve the quality of life with aging.

### REFERENCES

Abraham J Domb, Zeev Zehavi. (1999). U.S. Pat. No. 6,365,173 B1.

Arya, A., Abdullah, M. A., Haerian, B. S., & Mohd, M. A. (2012). Screening for Hypoglycemic Activity on The Leaf Extracts of Nine Medicinal Plants: In-Vivo Evaluation. E-Journal of Chemistry, 9(3), 1196-1205.

Bohlender, J. M., Franke, S., Stein, G., & Wolf, G. (2005). Advanced glycation end products and the kidney. Am J Physiol Renal Physiol, 289(4), F645-659. doi: 289/4/F645 [pii]

Deguchi, Y., & Miyazaki, K. (2010). Anti-hyperglycemic and anti-hyperlipidemic effects of guava leaf extract. Nutr Metab (Loud), 7, 9. doi: 10.1186/1743-7075-7-9

Flodin, N. W. (1997). The metabolic roles, pharmacology, and toxicology of lysine. J Am Coll Nutr, 16(1), 7-21.

Griffith, R. S., Walsh, D. E., Myrmel, K. H., Thompson, R. W., & Behforooz, A. (1987). Success of L-lysine therapy in frequently recurrent herpes simplex infection. Treatment and prophylaxis. Dermatologica, 175(4), 183-190.

Hernandez-Galicia, E., Aguilar-Contreras, A., Aguilar-Santamaria, L., Roman-Ramos, R., Chavez-Miranda, A. A., Garcia-Vega, L. M., . . . Alarcon-Aguilar, F. J. (2002). Studies on hypoglycemic activity of Mexican medicinal plants. Proc West Pharmacol Soc, 45, 118-124.

Kalogeropoulou, D., LaFave, L., Schweim, K., Gannon, M. C., & Nuttall, F. Q. (2009). Lysine ingestion markedly attenuates the glucose response to ingested glucose without a change in insulin response. Am J Clin Nutr, 90(2), 314-320. doi: 10.3945/ajcn.2008.27381

Kato, T., Sano, M., & Mizutani, N. (1987). Inhibitory effect of intravenous lysine infusion on urea cycle metabolism. Eur J Pediatr, 146(1), 56-58.

Lugg, J. W., & Weller, R. A. (1948). Partial amino-acid composition of some plant-leaf protein preparations; the arginine, histidine and lysine contents. Biochem J, 42(3), 408-412.

Mazak, K., Kokosi, J., & Noszal, B. (2011). Lipophilicity of zwitterions and related species: a new insight. Eur J Pharm Sci, 44(1-2), 68-73. doi: 10.1016/j.ejps.2011.06.009

Monnier, V. M., Sun, W., Sell, D. R., Fan, X., Nemet, I., & Genuth, S. (2014). Glucosepane: a poorly understood advanced glycation end product of growing importance for diabetes and its complications. Clin Chem Lab Med, 52(1), 21-32. doi: 10.1515/cclm-2013-0174

Mudra, M., Ercan-Fang, N., Zhong, L., Fume, J., & Levitt, M. (2007). Influence of mulberry leaf extract on the blood glucose and breath hydrogen response to ingestion of 75 g sucrose by type 2 diabetic and control subjects. Diabetes Care, 30(5), 1272-1274. doi: 10.2337/dc06-2120

Sensi, M., De Rossi, M. G., Celi, F. S., Cristina, A., Rosati, C., Perrett, D., . . . Di Mario, U. (1993). D-lysine reduces the non-enzymatic glycation of proteins in experimental diabetes mellitus in rats. Diabetologia, 36(9), 797-801.

Tome, D., & Bos, C. (2007). Lysine requirement through the human life cycle. J Nutr, 137(6 Suppl 2), 1642S-1645S. doi: 137/6/1642S [pii]

Umar, Z. U., Moh'd, A., & Tanko, Y. (2013). Effects of Ethanol Leaf Extract of Ficus Glumosa on Fasting blood Glucose and Serum Lipid Profile in Diabetic Rats. Niger J Physiol Sci, 28(1), 99-104.

Zhang, P., Zhang, X., Brown, J., Vistisen, D., Sicree, R., Shaw, J., & Nichols, G. (2010). Global healthcare expenditure on diabetes for 2010 and 2030. Diabetes Res Clin Pract, 87(3), 293-301. doi: 10.1016/j.diabres.2010.01.026

Having described my invention, I claim:

1. A method to lower blood glucose and decrease advanced glycation end products (AGEs) by administration of l-lysine.

2. The method of claim 1 where l-lysine is incorporated within a stereocomplex matrix and administered as a sustained release preparation.

3. A method to lower blood glucose and decrease advanced glycation end products (AGEs) by administration of enantiomeric mixtures of lysine.

4. The method of claim 3 where enantiomeric mixtures of lysine are incorporated within a stereocomplex matrix and administered as a sustained release preparation.

5. A method to competitively inhibit formation of advanced glycation end product crosslinks in the presence of d-enantiomers of advanced glycation end products (AGEs).

6. The method of claim 5 where the d-enantiomers of AGEs are comprised of covalent bonds linking d-amino acids.

7. The method of claim 5 where the d-enantiomers of AGEs are comprised of covalent bonds linking d-lysine.

8. The method of claim 5 where the d-enantiomers of AGEs are comprised of AGE free adducts.

9. A method to competitively inhibit activation of advanced glycation end product receptors (RAGE) in the presence of d-enantiomers of advanced glycation end products (AGEs).

10. The method of claim 8 where the d-enantiomers of AGEs are comprised of covalent bonds linking d-amino acids.

11. The method of claim 8 where the d-enantiomers of AGEs are comprised of covalent bonds of d-lysine.

12. The method of claim 8 where the d-enantiomers of AGEs are comprised of AGE free adducts.
